(19) United States
(12) Patent Application Publication
(10) Pub. No.: US 2013/0143845 A1
(43) Pub. Date: Jun. 6, 2013

(54) VITAMIN D COMPOUNDS AND METHODS FOR ENHANCING MUSCLE STRENGTH, AND PREVENTION AND TREATMENT OF DISEASE IN HUMAN BEINGS

(71) Applicants: William Francis Supple, Shelburne, VT (US); Antoinette G. Supple, Shelburne, VT (US); Laura J. Supple, Shelburne, VT (US)

(72) Inventors: William Francis Supple, Shelburne, VT (US); Antoinette G. Supple, Shelburne, VT (US); Laura J. Supple, Shelburne, VT (US)

(21) Appl. No.: 13/310,999

(22) Filed: Dec. 5, 2011

(51) Int. Cl. A61K 31/593; C12N 5/09; C12N 5/078; C12N 5/077; C12N 5/079; A61P 21/00; A61P 3/00; A61P 17/00; A61P 29/00; A61P 17/14; A61P 17/06; A61P 37/06; A61P 1/00; A61P 25/00; A61P 3/10; C12N 5/071

(52) U.S. Cl. 514/167; 435/375

(57) ABSTRACT

Compositions and methods for enhancing muscular strength through the development of fast twitch skeletal muscle fibers comprising the steps of administering therapeutically effective amounts of Vitamin D, folic acid, calcium carbonate and potassium gluconate compounds to a subject, and the treatment and prevention of disease in human beings.

### CROSS-REFERENCE TO RELATED APPLICATIONS

Not Applicable.

### STATEMENT OF FEDERALLY SPONSORED RESEARCH

Not Applicable.

### FIELD OF THE INVENTION

The present invention relates to compounds and methods for increasing muscular strength and measures of athletic performance, and the prevention and treatment of various acute and chronic diseases in subjects.

### BACKGROUND OF THE INVENTION

There are two types of skeletal muscle commonly classified as slow-twitch and fast-twitch muscle fiber. Slow-twitch fibers (or type I) fibers are relatively slow to reach full contraction whereas fast-twitch muscle fiber (type II) contracts very rapidly, individual fast-twitch fibers are much larger in size than slow-twitch fibers hence they are more powerful. Athletic behaviors that entail ballistic, powerful and short latency responses are potentiated in athletes whose muscles are preferentially comprised of fast-twitch fibers.

Almost every cell in the body has a receptor for Vitamin D, hence the growing appreciation that vitamin D is involved in many biological processes, not just calcium homeostasis and bone maintenance. Every skeletal muscle cell has Vitamin D receptors. More than 1000 human genes are direct targets of 1,25(OH)₂D₃ (the active form of vitamin D) including the genes involved in the production of muscle. 1,25(OH)₂D₃ affects the growth of new muscle and affects the performance of existing muscle. Bischoff, et al (2001) reported the first in situ detection of the VDR in human skeletal muscle via intranuclear staining of the 1,25(OH)₂D₃ receptor. Once within the nucleus, 1,25(OH)₂D₃ binds to its receptor initiating changes in gene transcription of mRNA and subsequent de novo protein synthesis (Freedman, 1999). Birge and Haddad (1975) found that exogenous vitamin D affects protein synthesis in muscle.

Muscle fibers show a remarkable ability to change their response characteristics during certain types of training. However, these changes appear to be haphazard and variable as the conditions governing optimal use-dependent plasticity are unknown (Gollnick, 1972). Slow-twitch fibers can acquire fast-twitch fiber characteristics, and fast-twitch can transform into slow-twitch to a lesser extent. Skeletal muscle fibers do not exist in separate forms at the subcellular level but are distributed along a continuum based on the multitude of combinations of myosin heavy and light chain isoforms, polymorphic expression of protein isoforms, metabolic potential and calcium handling characteristics (Gollnick et al., 1972). These features enable muscle cells to adapt and to change based on need and to exhibit some degree of plasticity in response to training (Gollnick et al., 1973). However, the conditions under which optimal plasticity is achieved unknown. It is discovered that the present invention provides conditions under which use-dependent muscle fiber plasticity is robustly induced and controlled in a variety of subject populations.

Human skin makes vitamin D when exposed to ultraviolet energy (UVB) in sunlight. Despite the fact that Vitamin D₃ is readily available via cutaneous synthesis, vitamin D deficiency is epidemic throughout the population because of sun avoidance behavior, use of sunscreens and living in northern latitudes. Estimates of deficiency range from 90% for older populations to 70% deficiency in younger, more active populations (Gordon et al., 2004). Latitude, age, skin pigmentation, obesity and air quality all effect the ability of the sun to generate 25(OH)D₃ in skin. Most people of all age groups are vitamin D deficient. Athletes that train indoors, live in the north (>35° latitude), and/or have dark skin are those most likely to be severely vitamin D deficient. Even those living in sunny lower latitudes are at risk of deficiency if they consciously avoid the sun and/or use sunblock. Many groups have been found to have severe vitamin D deficiency despite living in areas of high ambient sunshine including active adults in Miami (Levis et al., 2005), inner city youths in the US (Gordon, 2004), elite gymnasts in Australia (Lovell, 2008), young Hawaiian skateboarders (Binkley et al., 2007), professional basketball players in Spain (Garcia & Guisado, 2011) and adolescent girls in England (Ward et al., 2009).

Athletes with dark skin face additional problems. Melanin acts as an effective sunscreen, as athletes with a high concentration of melanin in their skin need up to 10 times longer UVB exposure to generate the same vitamin D stores that do lighter-skinned people (Holick, 2007). For example, African-American professional basketball players had serum 25(OH)D₃ levels approximately half of those of their white counterparts even though they consumed a similar diet (Garcia & Guisado, 2011).

Athletes would be expected to eat well yet many studies show that they take in very little vitamin D. Large cross-sectional studies found that vitamin D deficiency is common in otherwise apparently healthy adults (e.g. Chapuy et al., 1997). For example, despite consuming high-energy diets (almost 18,000 calories/day), professional basketball players only gained about 140 IU vitamin D₃/day from dietary sources (Garcia & Guisado, 2011). The average dietary vitamin D intake in adolescents and young adults in the United States (from milk, other fortified foods, fish, and supplements combined) is 200-300 IU/day (Yetley, 2008). This dietary intake is too low to have significant effects on serum 25(OH)D levels (Vieth, 1999). These chronic low levels result in vitamin D starvation causing all the available vitamin D to be diverted for use for the body's immediate metabolic needs (first-order mass action kinetics) (e.g. Hollis et al., 2007). No vitamin D can be stored in the fat or muscle for later use until serum levels of 25(OH)D reach 45-50 ng/ml or above for sustained periods of time.

The present invention solves the problems associated with other high-dosage vitamin D products through a novel manipulation of the pharmacokinetic time course of the breakdown and subsequent deactivation of 1,25(OH)₂D₃. As is the case with most steroid hormones, the effect of the hormone 1,25(OH)₂D₃ is limited, restricted, truncated and/or terminated by metabolic processes. Manipulation of these processes can have the effect of potentiating the active effects of 1,25(OH)₂D₃. Folic acid blocks the gene producing the enzyme (CYP24A₁) that catabolizes 1,25(OH)₂D₃ (Cross et al., 2006). In essence, folic acid functions as an agonist of the active form of vitamin D, 1,25(OH)₂D₃ by delaying it's degradation. In the case of fast twitch muscle fiber, the folate in the invention extends the window of time, from about 4 hours (Holick, 2003) to about 12 hours, during which 1,25(OH)₂D₃ actively initiates gene transcription, encoding the development of fast-twitch muscle fibers and resultant protein synthesis (Lichtmann, personal communication).

Physical and athletic performance is seasonal; peaking during the late summer months and decrementing in winter (e.g. Cannell et al., 2009). In the early 20th century athletes were using UVB rays as an ergogenic aid (e.g. Gorkin et al., 1938) however, these studies were simply observational as no mechanisms were proposed to explain the observed effects, or, more importantly, how they might be controlled and manipulated. The examples presented here concerning the present invention are the first to systematically demonstrate that manipulation of vitamin D₃ results in significant performance effects in a variety of common sport-related motor tasks. No invention exists composed of vitamin D₃, folic acid, calcium carbonate and potassium gluconate that increases muscular strength, increases fast-twitch muscle fiber, measures of athletic performance and/or prevents and treats disease in subjects. Therefore the need exists for a new class of effective vitamin D₃ compounds that control muscular strength, performance and function, and prevent and treat disease. Furthermore, the present invention increases muscular strength and measures of athletic performance without causing undesirable side effects associated with other performance enhancing substances such as anabolic steroids, pharmaceuticals or stimulants. Quite to the contrary, the present invention has been shown to have beneficial effects on the amelioration and prevention of various acute and chronic diseases in subjects.

The invention is identified and referred to as D₃F_{A}C_{C}P_{G}. D₃F_{A}C_{C}P_{G} is a novel formulation useful according to the subject invention, and proven safe and beneficial for humans. In a preferred embodiment, D₃F_{A}C_{C}P_{G} comprises vitamin D₃, folic acid, calcium carbonate and potassium gluconate. D₃F_{A}C_{C}P_{G} can be used according to the subject invention as a nutritional supplement to enhance athletic performance and prevent disease. The benefits of D₃F_{A}C_{C}P_{G} include at least the following:
1) Enhanced, more powerful overhand throwing performance as measured by the increased velocity of a pitched baseball compared to baseline measures, in the same subjects, after administration of D₃F_{A}C_{C}P_{G}.
2) Enhanced, more powerful sprint running performance as measured by the decreased time to run a 40 yard sprint compared to baseline measures, in the same subjects, after administration of D₃F_{A}C_{C}P_{G}.
3) Enhanced, more powerful standing vertical jump performance as measured by the increased height of vertical jump compared to baseline measures, in the same subjects, after administration of D₃F_{A}C_{C}P_{G}.
4) Enhanced, more powerful tennis serve velocity as measured by the increased velocity of a overhand served tennis ball compared to baseline measures, in the same subjects, after administration of D₃F_{A}C_{C}P_{G}.
5) Enhanced, more powerful leg press performance as measured by the increased amount of weight lifted compared to baseline measures, in the same subjects, after administration of D₃F_{A}C_{C}P_{G}.
6) Increased type II fast twitch skeletal muscle fiber in affected muscles, leading to faster, more forceful movements.
7) Reduction of body weight as measured by the decreased weight of subjects compared to baseline measures after administration of D₃F_{A}C_{C}P_{G}.
8) D₃F_{A}C_{C}P_{G} is a safe means to optimize and enhance athletic performance without the harmful side effects or illegality of anabolic steroids, pharmaceuticals or stimulants.
9) In athletes, the significant muscle performance gain attributable to D₃F_{A}C_{C}P_{G} can provide a winning edge among nearly equal competitors.
10) Due to the unique mechanism of action D₃F_{A}C_{C}P_{G} affects all biological processes affected, controlled, influenced, modified, attenuated, inhibited and/or potentiated by vitamin D₃, and has been shown to have beneficial effects on the amelioration and prevention of various acute and chronic diseases in subjects.
   No prior art patents disclose the nutritional composition of the present invention for enhancing athletic performance, muscular performance, fast twitch muscle fiber growth or disease treatment or prevention.

It is not intended that the present invention be limited to a particular mechanism of action. Indeed, an understanding of the mechanism is not necessary to make and use the present invention. However, insufficient sunlight for vitamin D₃ biosynthesis is a performance hindering and disease determining environmental risk factor. As such, the therapeutic effect of the administration of biologically active vitamin D₃ compounds to subjects may be achieved by compensating for insufficient vitamin D₃ biosynthesis in certain subjects. This invention functions to restore a deficiency, and then maintain optimized 1,25(OH)₂D₃ levels to effect gene-transcripted, use-dependent fast-twitch muscle fiber plasticity.

Without intending to be bound or limited by theory, it is believed that the present invention containing the vitamin D₃, folic acid, calcium carbonate and potassium gluconate formulation functions to synergistically prolong the half-life of the active steroidal hormone 1,25(OH)₂D₃ from approximately 4 hours (Holick, 2003) to approximately 12 hours in circulation and at target receptors in all tissues with vitamin D receptors. Folic acid blocks the gene producing the enzyme (CYP24A₁) that catabolizes 1,25(OH)₂D₃. For example, in the case of fast twitch muscle fiber, the folate in the invention extends the window of time from about 4 hours to about 12 hours during which 1,25(OH)₂D₃ actively initiates gene transcription, encoding the development of fast-twitch muscle fibers and resultant protein synthesis.

As such, the invention, by altering the effective half-life of the steroidal hormone 1,25(OH)₂D₃ and its active period at the appropriate receptor, will affect all biological and physiological processes controlled, influenced, modified, arrested and mediated by vitamin D and its metabolites. The present invention is effective in preventing and/or curing the incidence of diseases and disorders including but not limited to psoriasis, colitis, obesity, diabetes, neoplastic diseases of epithelial origin (cancer of the skin, prostate, breast, lung and colon), depression, autism, respiratory tract infections, asthma, autoimmune diseases such as multiple sclerosis, cardiovascular disease, influenza, osteoporosis, osteoarthritis, atheriosclerosis, multiple sclerosis, fibromylagia, alopecia, systemic and vascular inflammation, concussion and aging.

So that the matter in which the above-recited features, advantages and objects of the invention, as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrated preferred embodiments of the invention and therefore are not to be considered limiting in their scope.

### SUMMARY OF THE INVENTION

The present invention provides a method of enhancing muscular power and energy as measured by standing vertical jump, running sprint speed, thrown baseball velocity, tennis ball serve velocity and leg press strength, and preventing and/or curing the incidence of diseases and disorders including but not limited to psoriasis, colitis, obesity, diabetes, neoplastic diseases of epithelial origin (cancer of the skin, prostate, breast, lung and colon), depression, autism, respiratory tract infections, asthma, influenza, osteoporosis, osteoarthritis, atheriosclerosis, multiple sclerosis, fibromylagia, alopecia, systemic and vascular inflammation, concussion and aging.

The following examples are given for the purpose of illustrating various embodiments of the invention and are not meant to limit the present invention in any fashion.

The invention is identified and referred to as D₃F_{A}C_{C}P_{G}. And when referring to the invention (D₃F_{A}C_{C}P_{G}) we mean any compound or derivative compound of the vitamin-D₃ composition described above including, but not limited to, a composition comprising a first amount of a biologically active vitamin D₃ compound cholecalciferol, a second amount of folic acid, a third amount of calcium carbonate and a fourth amount of potassium gluconate or pharmaceutical compositions thereof.

It should be noted that the above description, attached figures and their descriptions are intended to be illustrative and not limiting of this invention. Many themes and variations of this invention will be suggested to one skilled in this and, in light of the disclosure. All such themes and variations are within the contemplation thereof. For instance, while this invention has been described in conjunction with the various exemplary embodiments outline herein, various alternatives, modifications, variations, improvements, and/or substantial equivalents, whether known or that rare or may be presently unforeseen, may become apparent to those having at least ordinary skill in the art. Various changes may be made without departing from the spirit and scope of the invention. Therefore, the invention is intended to embrace all known or later-developed alternatives, modifications, variations, improvements, and/or substantial equivalents of these exemplary embodiments.

### BRIEF DESCRIPTION OF THE DRAWINGS

For all the drawings the values associated with the data points within the Figures, either line graphs or histograms, represent the means for the relevant group.

FIG. 1 depicts the mean velocity of a pitched baseball in miles per hour (mph) in a within-subjects experiment with young male subjects (n=24). In sequence, under baseline conditions before any treatment, after the first 90 day D₃F_{A}C_{C}P_{G} treatment (TEST 1), after 90 day placebo treatment (TEST 2) and following another 90 day D₃F_{A}C_{C}P_{G} treatment (TEST 3). The inset shows the average increase in MPH and percentage change from baseline to TEST 1.

FIG. 2 depicts the mean velocity of a pitched baseball in miles per hour (mph) in a within-subjects experiment (n=24), in sequence, under baseline conditions before any treatment, after 90 day placebo treatment (TEST 1), after a 90 day D₃F_{A}C_{C}P_{G} treatment (TEST 2) and following another after 90 day placebo treatment (TEST 3). The inset shows the average increase in MPH and percentage change from placebo to TEST 2.

FIG. 3 depicts the mean 40 yard dash time in seconds (sec) in a within-subjects experiment (n=24), in sequence, under baseline conditions before any treatment, after the first 90 day D₃F_{A}C_{C}P_{G} treatment (TEST 1), after 90 day placebo treatment (TEST 2) and following another 90 day D₃F_{A}C_{C}P_{G} treatment (TEST 3).

FIG. 4 depicts the mean 40 yard dash time in seconds (sec) in a within-subjects experiment (n=24), in sequence, under baseline conditions before any treatment, after 90 day placebo treatment (TEST 1), after a 90 day D₃F_{A}C_{C}P_{G} treatment (TEST 2) and following another after 90 day placebo treatment (TEST 3).

FIG. 5 depicts the mean standing vertical jump height (in inches) in a within-subjects experiment (n=24), in sequence, under baseline conditions before any treatment, after the first 90 day D₃F_{A}C_{C}P_{G} treatment (TEST 1), after 90 day placebo treatment (TEST 2) and following another 90 day D₃F_{A}C_{C}P_{G} treatment (TEST 3). The inset shows the average increase in jump height and percentage change from baseline to TEST 1.

FIG. 6 depicts the mean standing vertical jump height (in inches) in a within-subjects experiment (n=24), in sequence, under baseline conditions before any treatment, after 90 day placebo treatment (TEST 1), after a 90 day D₃F_{A}C_{C}P_{G} treatment (TEST 2) and following another after 90 day placebo treatment (TEST 3). The inset shows the average increase in jump height and percentage increase from placebo to TEST 2.

FIG. 7 a summary figure that depicts the average increase in pitch velocity (mph), sprint speed (sec), and standing vertical jump (inches) after the first D₃F_{A}C_{C}P_{G} treatment combining the data from both groups (n=48). Shown is the mean percentage change in performance. The average percentage change of each measure is shown in the bar. The average baseline-to-first D₃F_{A}C_{C}P_{G} test measures are shown above the corresponding bar as well as the mean change in mph, sec and inches for the associated measures.

FIG. 8 depicts the mean standing vertical jump height (inches) in a within-subjects experiment (n=38) in older male subjects. In sequence, under baseline conditions before any treatment, after the first 90 day D₃F_{A}C_{C}P_{G} treatment, after 90 day placebo treatment and following another 90 day D₃F_{A}C_{C}P_{G} treatment. The inset shows the average percentage increase from baseline to the first D₃F_{A}C_{C}P_{G} test.

FIG. 9 depicts the mean standing vertical jump height (inches) in a within-subjects experiment (n=45) in older female subjects. In sequence, under baseline conditions before any treatment, after the first 90 day D₃F_{A}C_{C}P_{G} treatment, after 90 day placebo treatment and following another 90 day D₃F_{A}C_{C}P_{G} treatment. The inset shows the average percentage increase from baseline to the first D₃F_{A}C_{C}P_{G} test.

FIG. 10 depicts the mean one repetition maximum (1RM) leg press performance (normalized for presentation purposes multiplying by 100) and tennis ball serve velocity (mph) for middle-aged male tennis players (n=20), under baseline conditions before any treatment, after the first 90 day D₃F_{A}C_{C}P_{G} treatment, after 90 day placebo treatment and following another 90 day D₃F_{A}C_{C}P_{G} treatment. The top inset shows the average percentage increase in tennis serve velocity from baseline to the first D₃F_{A}C_{C}P_{G} test. The bottom inset shows the average percentage increase in 1RM leg press from baseline to the first D₃F_{A}C_{C}P_{G} test.

FIG. 11 depicts tennis ball serve velocity in four temporarily injured subjects (n=4) under baseline conditions before any treatment, after the first 90 day D₃F_{A}C_{C}P_{G} treatment, after 90 day placebo treatment and following another 90 day D₃F_{A}C_{C}P_{G} treatment. The inset marks the 90 day period of injury which prevented normal physical activity during the first phase of the experiment. The values associated with each data point are the individual tennis serve velocities for each subject.

FIG. 12 depicts 1RM leg press strength in four temporarily injured subjects (n=4) under baseline conditions before any treatment, after the first 90 day D₃F_{A}C_{C}P_{G} treatment, after 90 day placebo treatment and following another 90 day D₃F_{A}C_{C}P_{G} treatment. The inset marks the 90 day period of injury which prevented normal physical activity during the first phase of the experiment. The values associated with each data point is the individual leg press performance for each subject.

FIG. 13 is a summary figure that depicts the mean raw score (inside the bar) and mean percentage change (inset above the bar) from baseline after the first 90 day treatment with D₃F_{A}C_{C}P_{G} for tennis serve velocity (mph), leg press (1RM×100), standing vertical jump (inches) and body weight (pounds) in middle-aged males (n=20).

FIG. 14 depicts tennis serve velocity (mph) for each subject at baseline and after the first D₃F_{A}C_{C}P_{G} treatment. The values associated with each data point are the individual tennis serve velocities for each subject.

FIG. 15 depicts the “inverted-U” shaped dose response function effect of varying the concentration of vitamin D in D₃F_{A}C_{C}P_{G} on standing vertical jump height. Shown is the mean baseline jump height before treatment for each group (n=10) and jump height after treatment with various concentrations of vitamin D.

FIG. 16 depicts the effect of various combinations of the elements of the invention on standing vertical jump performance in young adult males. Shown is the mean±SEM baseline jump height and the jump height at the test after 90 days of daily treatment with placebo, D₃F_{A}C_{C}P_{G}, D₃F_{A}, D₃C_{C} and D₃P_{G}. The amount of each element separately was D₃ 5150 IU (cholecalciferol), F_{A} 400 mcg (folic acid), C_{C} 600 mg (calcium carbonate), and P_{G} 60 mg (potassium gluconate) in each of the various compounds.

### DETAILED DESCRIPTION OF THE INVENTION

### I. In General

As used herein, the term “a” or “an”, when used in conjunction with the term “comprising” in the claims and/or the specifications, may refer to “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition herein can be implemented with respect to any other method or composition described herein.

As used herein, the term “or” in the claims refers to “and/or” unless explicitly indicated to refer to alternatives only or the alternatives are mutually exclusive, although the disclosure supports a definition that refers to only alternatives and “and/or.”

As used herein, the terms “effective amount,” “therapeutically effective” or “pharmacologically effective amount” are interchangeable and refer to an amount that results in a desired effect, a delay or prevention of onset of the cell proliferation and/or pathophysiological condition or results in an improvement or remediation of the symptoms of the same. As used herein, the term “inhibit” refers to the ability of the steroidal compounds described herein, to block, partially block, interfere, decrease, reduce or deactivate enzymes associated with the metabolism and/or deactivation of 1α,25-dihydroxyvitamin D₃ (1,25(OH)₂D₃). As used herein, for example, the term “stimulate” refers to the ability of the steroidal compounds to increase differentiation of cells. The steroidal compounds described herein are effective as both inhibitor and stimulator compounds. Those of skill in the art understand that the effective amount may improve the subject's condition, but may not be a complete cure of the disease, disorder and/or condition.

As used herein, the term “subject” refers to any target of the treatment.

The compound may be applied to the subject daily. In one preferred embodiment the invention is ingested in tablet form, once per day, in the morning with food. Other embodiments may involve a distributed dosing protocol where equivalent dosages of the invention are administered at defined intervals through a 24-hour period.

The composition of the invention can be formulated in any other suitable manner. For example, water-based solution suspensions can be formulated to deliver the invention in the embodiment of a ‘sports drink, smoothie shake, frozen confection,’ or in solid forms such as an ‘energy bar,’ cereal, or other food stuff.

The following examples are given for the purpose of illustrating various embodiments of the invention and are not meant to limit the invention in any fashion.

So that the matter in which the above-recited features, advantages and objects of the invention, as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

### II. The Invention

By D₃F_{A}C_{C}P_{G} it is meant the vitamin D₃ compound described above comprising a first amount of a biologically active vitamin D₃ compound cholecalciferol (5150 IU), a second amount of folic acid (400 mcg), a third amount of calcium carbonate [36% calcium] (600 mg), and a fourth amount of potassium gluconate [16% potassium] (60 mg) or pharmaceutical compositions thereof.

In a preferred embodiment the composition of this invention may be prepared as a dry powder, formed into a gelatin-coated tablet weighing about 840 mg comprising the following:

| | | | |
|---|---|---|---|
| Vitamin D₃ | (cholecalciferol) | 5150 | IU |
| Calcium carbonate | (36% calcium) | 600 | mg |
| Folic acid | (10% tituration) | 400 | mcg |
| Potassium gluconate | (16% potassium) | 60.0 | mg |
| Cellulose | | 63.5 | mg |
| Stearic acid | (vegetable grade) | 30.0 | mg |
| Sodium carboxymethylcellulose | | 20.0 | mg |
| Magnesium stearate | (vegetable grade) | 6.0 | mg |
| Silicon dioxide | | 5.0 | mg |

Kits. In an alternate embodiment of the invention, a kit for conducting methods of the present invention is provided. In one embodiment, the kit comprises a D₃F_{A}C_{C}P_{G} compound according to the present invention and instructions for use.

By “instructions for use” it is meant a publication, a recording, a diagram, or any other medium of expression which is used to communicate the usefulness of the invention for one of the purposes set forth herein. The instructional material of the kit can, for example, be affixed to a container which holds the present invention or be shipped together with a container which contains the invention. Alternatively, the instructional material can be shipped separately from the container or provided on an electronically accessible form on an internet website with the intention that the instructions for use and the invention be used cooperatively by the recipient.

### Example 1

### Effects of D₃F_{A}C_{C}P_{G} on Pitched Baseball Velocity, Sprint Speed and Standing Vertical Jump in Young Adult Males

The Examples below disclose compounds and methods to enhance muscle strength and athletic performance, enhance body weight loss, and treat and prevent disease in subjects.

The speed at which one can overhand throw a baseball is related to the relative distribution of fast-twitch muscle fibers. Amateur baseball pitchers from various baseball leagues were tested in a within subjects repeated measures design experiment to assess the effects of D₃F_{A}C_{C}P_{G} on pitched baseball velocity. Table 1 shows the experimental design for Group 1 and Group 2.

Subjects: Forty-eight 18 to 20 year old (mean=19.2 years) males recruited from summer amateur baseball teams. All were in good health. The subjects were self-identified pitchers.

Instructions: Subjects were informed that the intent of the study was to examine the effects of a nutritional supplement on performance. Subjects received 90 day supplies of placebo or D₃F_{A}C_{C}P_{G} in coded containers. Coaches were unaware of treatment group assignment. 90 day treatment periods were selected based on the results of pilot studies and the work of Vieth et al., (2001) and Heaney et al., (2003) finding the achievement of steady-state levels of circulating vitamin D metabolites after 90 days treatment.

Measures: Average pitch speed (5 consecutive pitches) from regulation mound (60′6″ distance) thrown to a catcher either outside on a field or inside facility (in winter). Pitch velocity measured to nearest one-tenth of MPH at hand release using a tripod-fixed Juggs® radar gun. 40 yard sprint time was measured on rubberized track surfaces to the nearest 1/100 sec using a Tag Heuer® double infrared sensor system in a single trial. Standing vertical jump was recorded in a single trial as the difference between reach and maximum jump height. In all instances subjects were allowed suitable time to warm-up before the test and announced when they felt ready to perform.

Procedure: Subjects randomly assigned to one of two groups. In total, the experiment runs for 270 days. Baseline measures of pitch velocity, 40 yard sprint speed and standing vertical jump (SVJ) taken before any treatment. One half of subjects receive D₃F_{A}C_{C}P_{G} for 90 days, the other half receives placebo (coated sucrose tablet) for 90 days. Subjects instructed to observe usual training regimen. Record pitch velocity, sprint time and SVJ at first 90 day test. Next, the treatments were reversed such that the subjects receiving D₃F_{A}C_{C}P_{G} now receive placebo and visa versa. Record pitch velocity, sprint time and SVJ at second 90 day test. Finally, the subjects were reverted back to the original treatment condition for 90 more days. Pitch velocity, sprint time and SVJ was recorded at third 90 day test point.

Group 1 was tested for pre-treatment or BASELINE performance. This group then received 90 days of D₃F_{A}C_{C}P_{G} then tested at TEST 1. Then they received 90 days of placebo and tested again at TEST 2. Finally they received D₃F_{A}C_{C}P_{G} for another 90 day period and were tested again at TEST 3.

Group 2 was tested similarly but received just one D₃F_{A}C_{C}P_{G} treatment. First BASELINE performance was measured. Then they received 90 days of placebo treatment then tested at TEST 1. Then they received 90 days of D₃F_{A}C_{C}P_{G} and tested again at TEST 2. Finally they received placebo again for another 90 day period and were tested again at TEST 3.

### Results

### Pitched Baseball Velocity

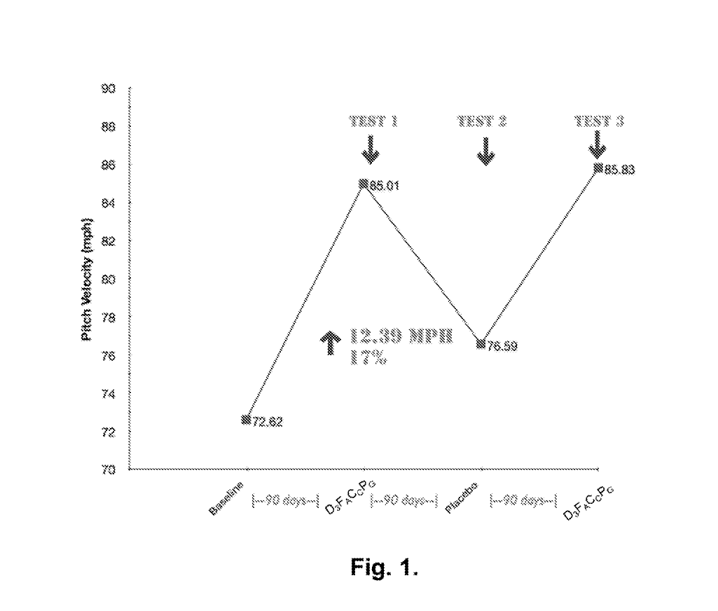

D₃F_{A}C_{C}P_{G} increases pitched/thrown baseball velocity. Group 1. FIG. 1. Baseline average pitch velocity was 72.62 mph. After 90 days of taking one D₃F_{A}C_{C}P_{G} tablet per day average pitch velocity increased approx. 13 mph to 85 mph. After removal of D₃F_{A}C_{C}P_{G} for 90 days, average pitch velocity decreased by 11 mph then reinstatement of D₃F_{A}C_{C}P_{G} for yet another 90 day period increased velocity to almost 86 mph. Each subject threw five pitches—there was no Trials effect indicating that there was no change in velocity during the 5 pitch sequence, therefore the mean of the 5 pitches was computed and used as a single data point. A one-way analysis of variance (ANOVA) for repeated measures was p<0.001, indicating an overall difference in the means. Individual t-tests found the effect of D₃F_{A}C_{C}P_{G} to be significant p<0.001, indicating an increase in velocity due to D₃F_{A}C_{C}P_{G} after the first 90 day period TEST 1. Velocity decreased significantly after the removal of D₃F_{A}C_{C}P_{G} under placebo TEST 2, p<0.001, and then greatly increased after the reinstatement of D₃F_{A}C_{C}P_{G} at the last data point TEST 3, p<0.001. A comparison of the means at Baseline and Test 2 was significantly different, with the velocity at Test 2 greater than at Baseline p=0.05.

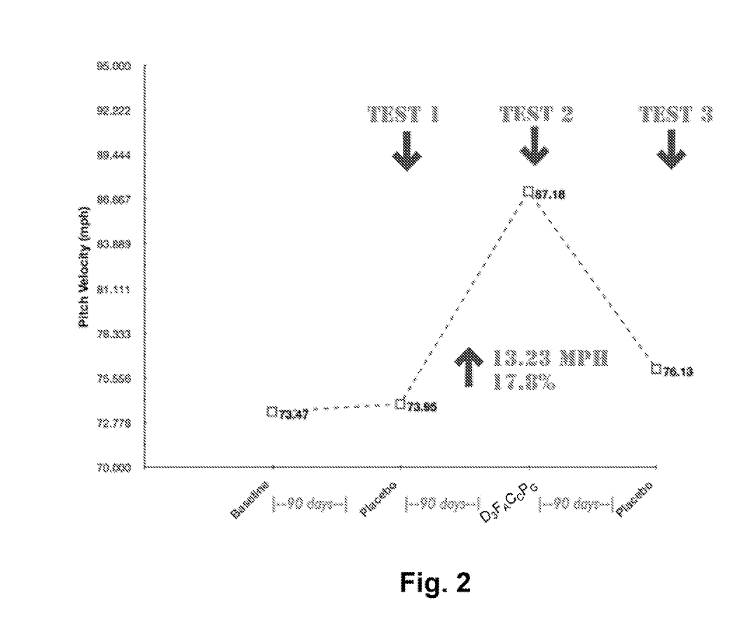

Results. FIG. 2. Group 2. Baseline average pitch velocity was 73.47 mph. This group received the placebo treatment for the first 90 days. There was no change in velocity from Baseline to Test 1, p>0.05. After 90 days of taking one D₃F_{A}C_{C}P_{G} tablet per day average pitch velocity increased 13.23 mph to 87 mph. After removal of D₃F_{A}C_{C}P_{G} for the next 90 days, average pitch velocity decreased by 11 mph to 76 mph. A one-way ANOVA for repeated measures was, p<0.001, indicating an overall difference in the means. Individual t-tests found the effect of D₃F_{A}C_{C}P_{G} to be significant p<0.001, indicating an increase in velocity due to D₃F_{A}C_{C}P_{G} after the second 90 day period TEST 2. Velocity decreased significantly after the removal of D₃F_{A}C_{C}P_{G} under placebo TEST 3, p<0.001. A comparison of the means at Baseline and Test 3 indicated a marginally significant difference, with the velocity at Test 3 greater than at Baseline p=0.06. This finding suggests that it may take more than 90 days for the effect of D₃F_{A}C_{C}P_{G} on fast-twitch fibers to dissipate under some conditions.

### 40 Yard Sprint

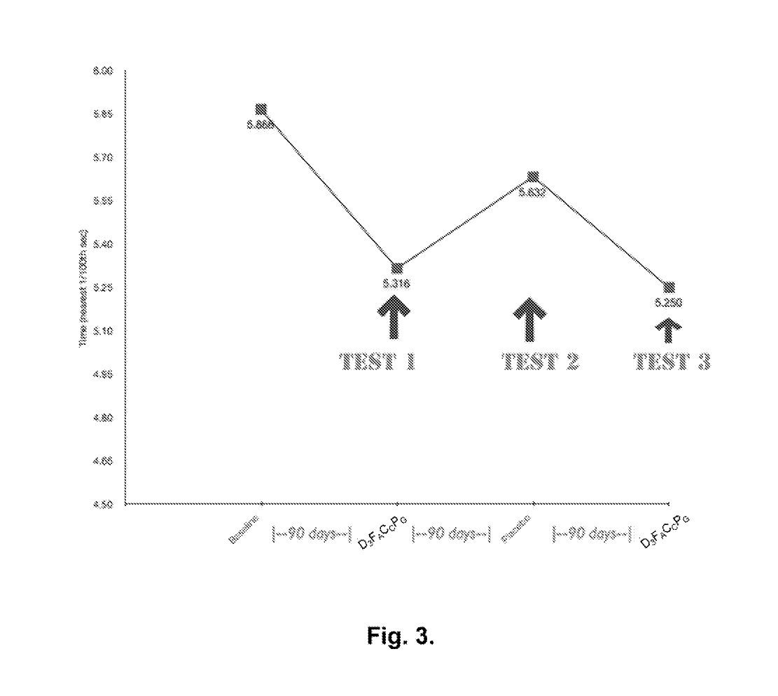

D₃F_{A}C_{C}P_{G} increases running sprint speed as measured by decreased time to run 40 yards. Group 1. FIG. 3. Baseline average 40 yard dash time was 5.86 sec. After 90 days of taking one D₃F_{A}C_{C}P_{G} tablet per day average time decreased 0.55 sec to 5.316 sec. After removal of D₃F_{A}C_{C}P_{G} for 90 days, average time increased by 0.316 sec then reinstatement of D₃F_{A}C_{C}P_{G} for yet another 90 day period decreased time to 5.25 sec. A one-way ANOVA for repeated measures was p<0.001, indicating an overall difference in the means. Individual t-tests found the effect of D₃F_{A}C_{C}P_{G} to be significant p<0.001, indicating a decrease in time (increase in speed) due to D₃F_{A}C_{C}P_{G} after the first 90 day period TEST 1. Time increased significantly after the removal of D₃F_{A}C_{C}P_{G} under placebo TEST 2, p<0.001, and then greatly decreased again after the reinstatement of D₃F_{A}C_{C}P_{G} at the last data point TEST 3, p<0.001.

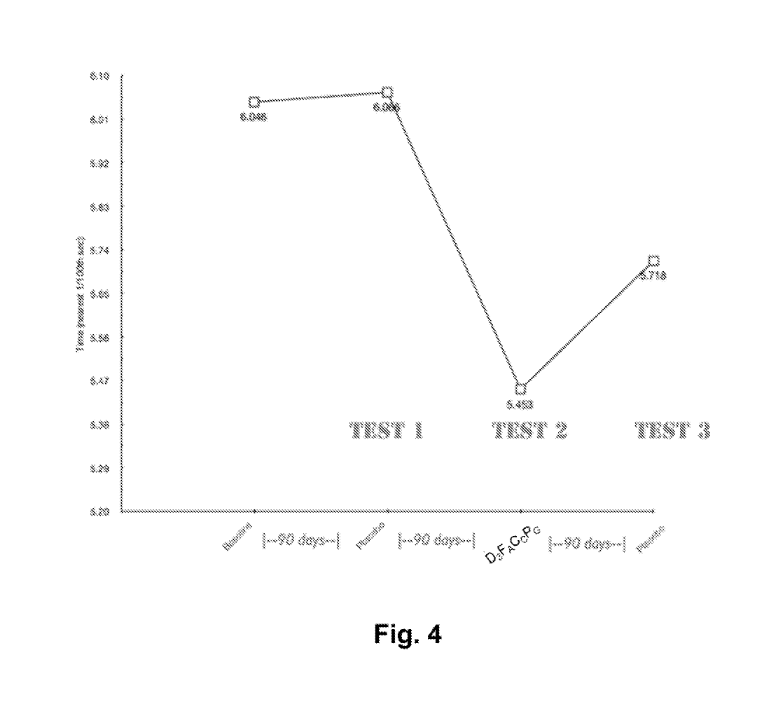

Results: Group 2, FIG. 4. Baseline average 40 yard dash time was 6.04 sec. After 90 days of taking one placebo tablet per day average time stayed the same increasing only 0.02 sec to 6.06 sec. Then taking D₃F_{A}C_{C}P_{G} for 90 days, average time decreased by 0.607 sec. Administration of placebo again reverted running times toward baseline, adding on average, 0.265 sec. A one-way ANOVA for repeated measures was significant, p<0.001, indicating an overall difference in the means. There was no significant difference between Baseline and Test 1 indicating that there was no placebo effect or maturation effect. Individual t-tests (Test 1 vs. Test 2) found the effect of D₃F_{A}C_{C}P_{G} to be significant, p<0.001, indicating a decrease in time (increase in speed) due to D₃F_{A}C_{C}P_{G} after the first 90 day period TEST 2. Time increased significantly after the removal of D₃F_{A}C_{C}P_{G} under placebo TEST 3, p<0.05.

### Standing Vertical Jump

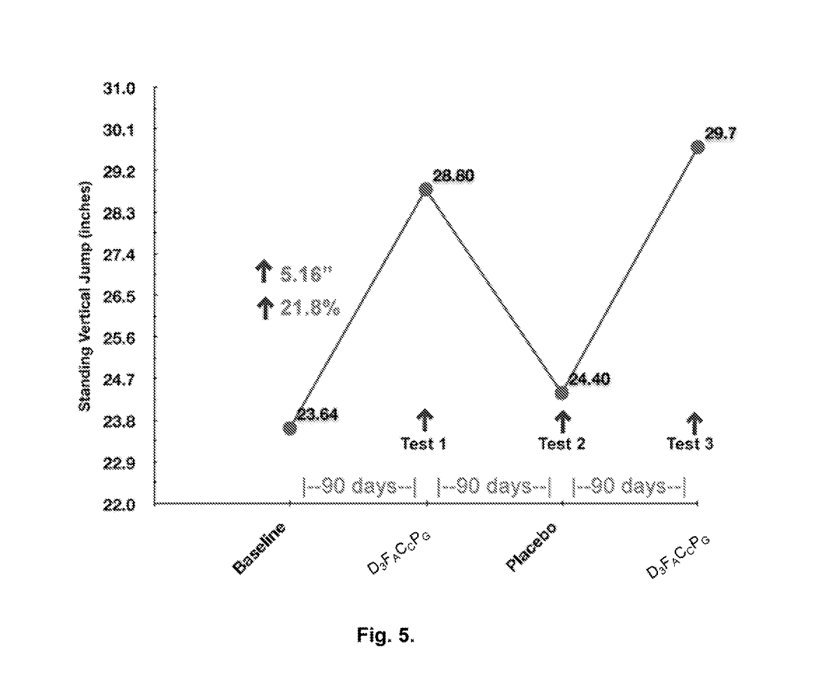

D₃F_{A}C_{C}P_{G} increases standing vertical jump height. Group 1. FIG. 5. Baseline average standing vertical jump (SVJ) was 23.64 inches. After 90 days of taking one D₃F_{A}C_{C}P_{G} tablet per day average SVJ increased 5.16 inches to 28.8 inches, a 21.8% increase. After removal of D₃F_{A}C_{C}P_{G} for 90 days, average SVJ decreased by 4.4 inches then reinstatement of D₃F_{A}C_{C}P_{G} for yet another 90 day period increased SVJ 5.3 inches to an average of 29.7 inches. All the apparent statistical differences are highly significant. A one-way ANOVA for repeated measures was p<0.001, indicating an overall difference in the means. Individual t-tests found the effect of D₃F_{A}C_{C}P_{G} to be significant p<0.001, indicating an increase in SVJ due to D₃F_{A}C_{C}P_{G} after the first 90 day period TEST 1. Subsequently, SVJ decreased significantly after the removal of D₃F_{A}C_{C}P_{G} under placebo TEST 2, p<0.001, and then greatly increased again after the reinstatement of D₃F_{A}C_{C}P_{G} at the last data point TEST 3, p<0.001.

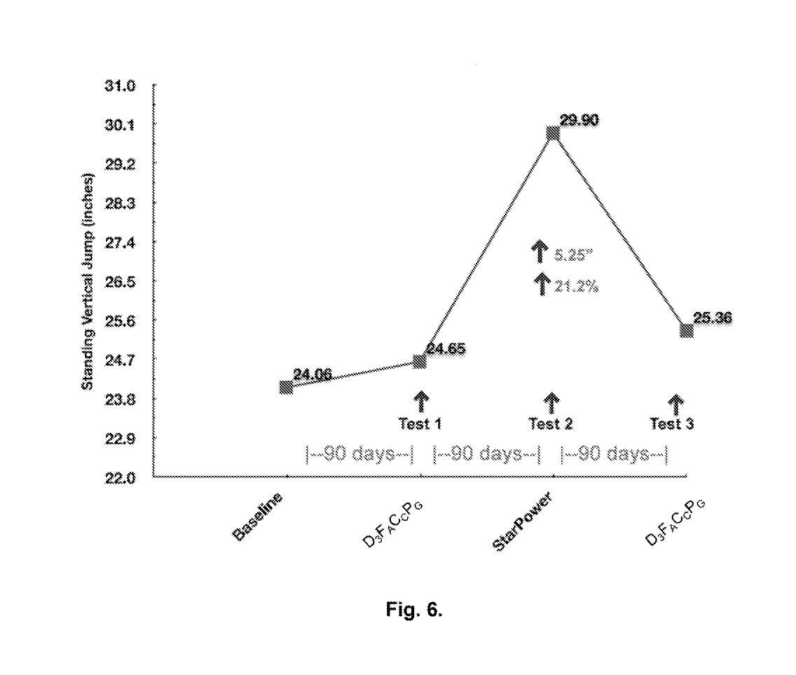

Results: Group 2. FIG. 6. Baseline average SVJ was 24.06 inches. After 90 days of taking one placebo tablet per day average SVJ stayed the same increasing only a fraction of an inch to 24.65 inches. Following administration of D₃F_{A}C_{C}P_{G} for 90 days, average SVJ increased by 5.25 inches to 29.9 inches—an increase of 21.2%. Administration of placebo again reverted SVJ toward baseline, resulting in a jump of 25.36 inches. All the apparent statistical differences were highly significant. A one-way ANOVA for repeated measures was significant, p<0.001, indicating an overall difference in the means. There was no significant difference between Baseline and Test 1 indicating that there was no placebo effect or maturation effect. Individual t-tests (Test 1 vs. Test 2) found the effect of D₃F_{A}C_{C}P_{G} to be significant, p<0.001, indicating an increase in SVJ due to D₃F_{A}C_{C}P_{G} after the first 90 day period TEST 2. SVJ then decreased significantly after the removal of D₃F_{A}C_{C}P_{G} under placebo TEST 3, p<0.01.

### Discussion

### Percentage Increase in Performance Following 90 day D₃F_{A}C_{C}P_{G} Treatment

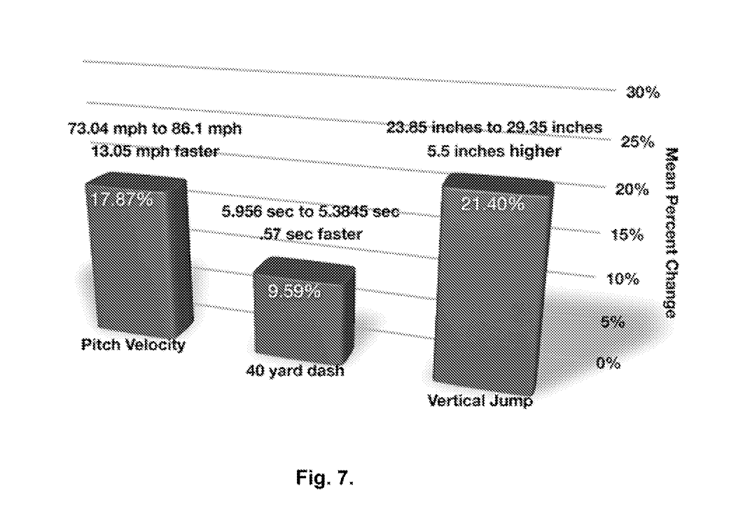

FIG. 7. combining all data of both groups following the first D₃F_{A}C_{C}P_{G} treatment, shows the average increase in performance.

Pitch Velocity: Before any treatment, subjects threw an average of 73 mph. After D₃F_{A}C_{C}P_{G}, they averaged 86 mph. The 13 mph increase represents a 17.87% increase in performance.

40 yard sprint time: Before any treatment, the average time was 5.9 sec. After D₃F_{A}C_{C}P_{G}, time to run 40 yards decreased to 5.38 sec. Overall, the improvement was 0.57 sec or a 9.59% change.

Standing Vertical Jump: Before treatment, subjects jumped 23.85 inches. After D₃F_{A}C_{C}P_{G} they now jumped 29.35 inches—an increase of 5.5 inches or a 21.4% increase.

### Example 2

### Effects of D₃F_{A}C_{C}P_{G} on Standing Vertical Jump in Middle-Aged Males and Females

To determine if there were effects that varied with sex and age, the effects of D₃F_{A}C_{C}P_{G} on standing vertical jump performance in middle aged males and females was examined.

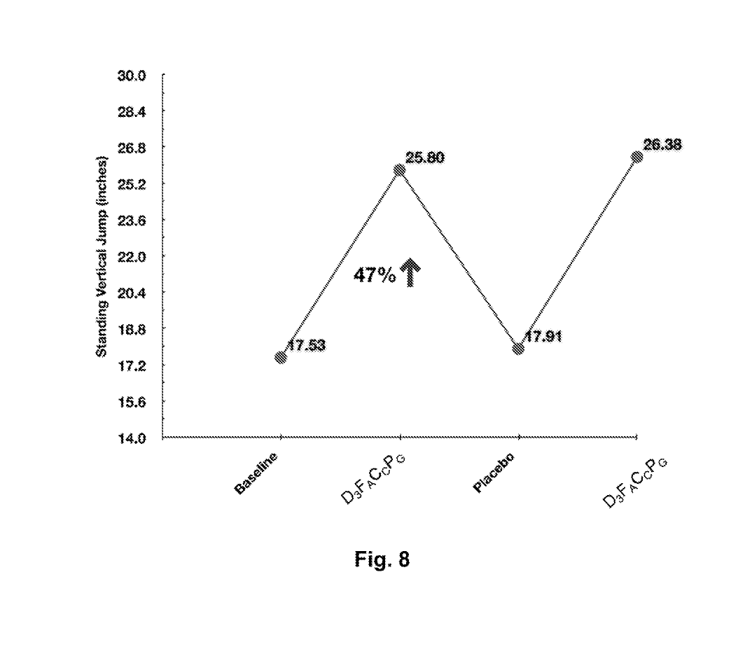

D₃F_{A}C_{C}P_{G} increases standing vertical jump in older males. The subjects were recruited by advertisement for a study examining the effects of nutritional supplements on athletic performance at health clubs. FIG. 8 presents the average SVJ performance using our within-subjects protocol. The average age of the subjects was 41 (range 38.2-44.5 yrs) and all were reported in good health. At baseline, the subjects averaged 17.53 inches (range 12-19.5 inches). After 90 days of D₃F_{A}C_{C}P_{G} treatment, average performance increased 8.27 inches to 25.8 inches (range 16.7-31.2 inches)—a 47% increase. A repeated measures ANOVA returned a significant Trials effect, F (3,37)=8.15, p<0.001 indicating a difference in recorded jump height among the treatment conditions. Planned t-tests among the means found that jump height was significant greater from Baseline after the first, p<0.005, and second, p<0.001, D₃F_{A}C_{C}P_{G} treatment.

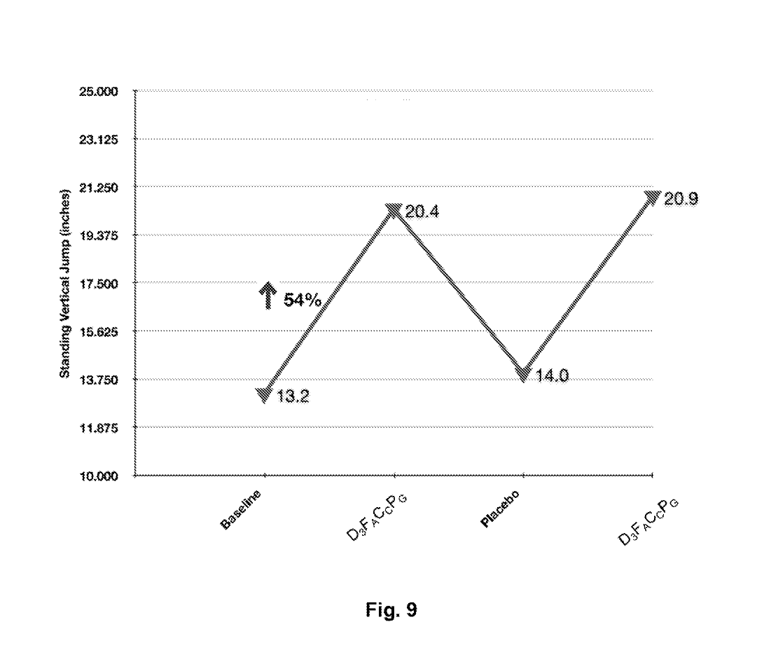

D₃F_{A}C_{C}P_{G} increases standing vertical jump in older males. Female subjects were recruited at the same time by advertisement for a study examining the effects of nutritional supplements on athletic performance at health clubs. FIG. 9 presents the average SVJ performance using our within-subjects protocol. The average age of the subject was 42.3 (range 34.5-47.5 yrs). At baseline, the subjects averaged 13.2 inches (range 8-24.3 inches). After 90 days of D₃F_{A}C_{C}P_{G} treatment, average performance increased 7.2 inches to 20.4 inches (range 12.5-28.5 inches)—a 54% average increase. Repeated measures ANOVA returned a significant Trials effect, p<0.001 indicating a difference in recorded jump height among the treatment conditions. Planned t-tests among the means found that jump height was significant greater from Baseline after the first, p<0.001, and second, p<0.001, D₃F_{A}C_{C}P_{G} treatment.

### Discussion

Sex Differences: D₃F_{A}C_{C}P_{G} is effective in increasing standing vertical jump in females indicating that there is no profound sex difference in the effectiveness of D₃F_{A}C_{C}P_{G} on standing vertical jump.

Age Differences: Compared to college-age subjects (which is the only comparison available) the overall magnitude of the potentiation in standing vertical jump in older subjects is greater. While not reaching the absolute heights observed in the younger subjects, the percentage change is much greater for the older subjects (47% for the 41 yr old males and 54% for the 42 yr old females) compared to the younger (˜21%). The baseline SVJ for the older males was 17.53 inches vs. 23.85 inches for the young males, a 6.32 inch difference between the two groups at the start. However, the absolute increase of 8.27 inches in the older vs. 5.2+ inches in the younger group under D₃F_{A}C_{C}P_{G} revealing a differential susceptibility to the effects of D₃F_{A}C_{C}P_{G} among these age groups.

### Example 3

### D₃F_{A}C_{C}P_{G} Increases Tennis Serve Velocity and Leg Strength in Middle Aged Males

This study examined the effects of D₃F_{A}C_{C}P_{G} on tennis serve velocity, leg strength and standing vertical jump in 35-51 year old male tennis players. The subjects were recruited through advertisement at several tennis clubs for a study the purpose of which was to examine the effects of nutritional supplements on sport performance. The caliber of the players was club level; the players were competitive within their leagues but older. Twenty-four subjects started the study. After instructions were given, subjects signed consent forms then height and weight measures were obtained. Baseline measures were taken next. First serve velocity was measured at the point of racquet impact using a Juggs® tripod-mounted radar gun. There was one trial per test session per subject. SVJ was measured in a single trial as described previously. Finally, leg strength was assessed on a Cybex® 5321 seated incline leg press machine using the protocol suggested by the American College of Sports Medicine to determine maximum leg strength—one repetition maximum (1RM). Subjects warmed up and estimated the maximum weight they could press. Successive approximations around that weight occurred with sufficient rest periods until the maximum leg press was determined. That weight was divided by the subject's body weight to control for body weight differences. Given the potential between- and within-machine variability in measured weight lifted, the same machine was used for all tests.

Procedure. Subjects were assessed before any treatment at Baseline. Subjects were instructed to take one tablet per day in the morning and to observe their usual routine; sport and otherwise. Subjects received D₃F_{A}C_{C}P_{G} for 90 days. At the end of this first 90 day period they were tested. Next placebo was administered for 90 days and another test occurred. Finally, the subjects were given D₃F_{A}C_{C}P_{G} for another 90 day period and tested again at the end of the 90 day period. Subjects did not know the design of the study nor the specific treatment they were given at any time.

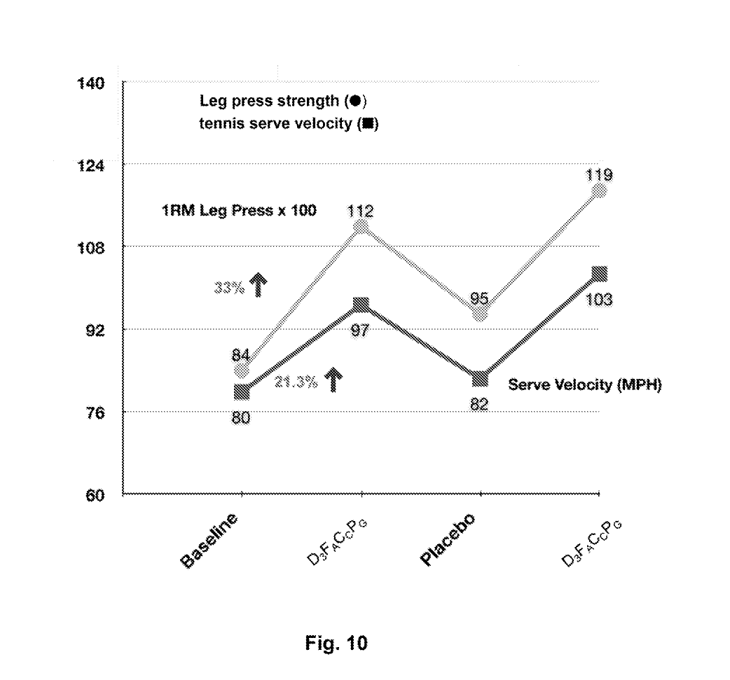

Results. Four subjects sustained injuries after baseline testing that prevented completion of the testing. Complete data are presented for twenty subjects in the FIG. 10. Mean baseline tennis serve speed was 80.2 mph (range 75.5-90.8 mph). Mean baseline 1RM (pounds successfully pressed/body weight) leg press was 0.84 (the value was multiplied by 100 for presentation purposes). The range was 0.69 to 1.21. These values indicate that these subjects could leg press between 69% and 121% of their body weight before treatment. After 90 days of D₃F_{A}C_{C}P_{G} treatment average serve velocity increased 17.2 mph to 97 mph (range 85.1-110.3 mph), a 21.3% increase. This change in velocity was significantly increased from baseline, p<0.01. 1 RM leg press strength increased 28 units (range 11-56), which translated into an average 33% increase in strength. Leg strength also was elevated from baseline, p<0.001. Removal of D₃F_{A}C_{C}P_{G} for the next 90 days returned performances to nearly baseline levels with serve velocity decreasing 15 mph to 82 mph and leg press performance decreasing significantly from the previous point, p<0.01 but still higher than baseline, p<0.05. In the last 90 day phase of the study D₃F_{A}C_{C}P_{G} was reintroduced and serve velocity increased again as did leg press strength, both effects were statistically significant p<0.001.

Examination of individual subject's data suggested a relationship between the two measures: velocity of the served ball and leg press strength in that the stronger players hit the ball harder. To control for absolute differences, percentage change measures were calculated for all data points with reference to the original baseline measures. The relationship between the percentage increase in velocity and percentage increase in leg press after the first 90 day D₃F_{A}C_{C}P_{G} treatment was r(19)=+0.78, p<0.01.

### Discussion

D₃F_{A}C_{C}P_{G} significantly increased tennis serve velocity and leg press performance in a group of middle-aged male club-level tennis players. The increase in velocity was notable for the group; an average of 17 mph or 21.3%. Leg strength was also greatly increased after taking D₃F_{A}C_{C}P_{G}; with an average 33% increase in leg press performance. Examination of the individual data revealed that every subject showed an increase in serve velocity and leg press strength and that the magnitude of the change in one measure (tennis serve) was positively correlated with the observed change in the other (leg press), r=+0.78.

As part of the debriefing procedure, subjects completed surveys regarding their experiences during the study. Subjects were informed which time periods corresponded with placebo and D₃F_{A}C_{C}P_{G} treatments. The vast majority of comments were favorable, relating to the positive hedonic state associated with enhanced physical performance. Comments such as “felt stronger,” “dominant,” “totally confident”, “happier,” “in control,” and “felt younger” reflected the overall tone of the responses. Negative comments pertained to the placebo phase of the study: displeasure with the loss of performance, frustration over the lack of control of treatment. Half of the subjects reported less soreness after a match, twelve felt they could either “see the ball better” and were “quicker to the spot.”

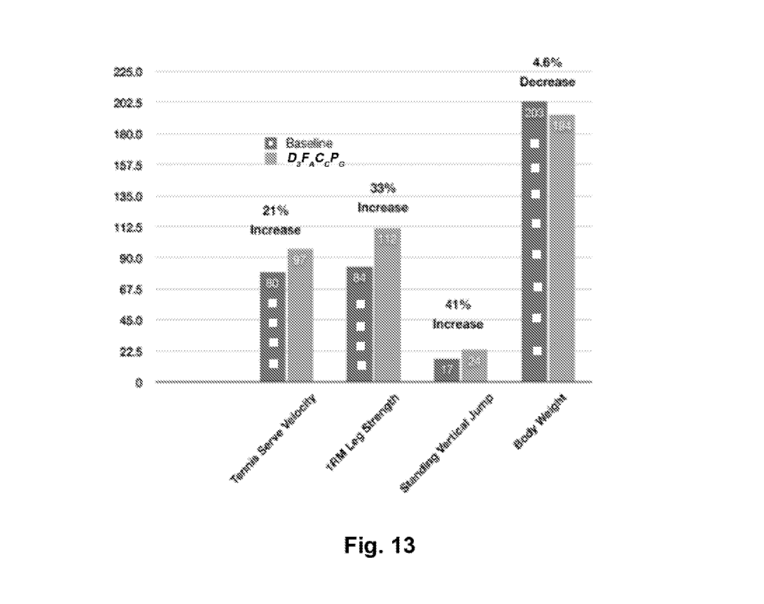

Weight Loss: Several subjects made statements to the effect, ‘I could tell that what you were testing worked, because I felt different when the treatment was switched.’ Subjects were also asked if they changed their work-out or training routines during the study. Eleven subjects reported having more energy, as a result they ‘went to the gym more.’ All twenty subjects reported losing weight over the course of the 270 day study. In an effort to quantify this observation, subjects were asked to submit to a re-weighing. Compared to initial baseline, subjects weighed between 4 and 27 lbs. less FIG. 13 (average 4.3%, range 0.02-10.8%). There was a positive correlation between initial body weight and magnitude of weight loss, with the heavier subjects losing the most weight, r(19)=+0.61, p<0.05. Since 1RM values would be even higher at the end of the study as the subjects lost weight throughout the period, revised values were not calculated as they would still be in the same direction and not change the conclusions.

### Example 4

### Dependence of Effect on Use-Dependent Plasticity. Four Injured Subjects

Four subjects completed the initial baseline measures were assigned treatment but were injured within two weeks. Two had ankle injuries, one a carpal tunnel issue and one a broken wrist; all injuries precluded any tennis related activity for 2+ months. Subjects continued to take the treatment during their convalescence except that they could not play tennis or engage in strenuous exercise. At 90 days, these four subjects had recovered, and were tested. Then completed the remaining phases of the study along with the 20 other subjects.

### Tennis Serve Velocity in Four Temporarily Injured Subjects

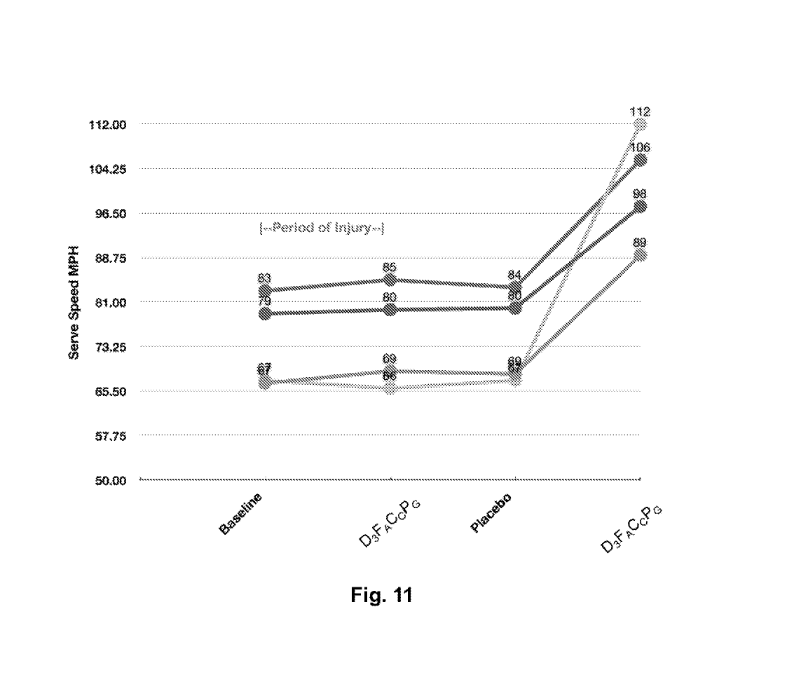

Serve Velocity Results: FIG. 11 depicts serve velocity measurements from each of the injured subjects. Baseline serve speed was similar to the rest of the group (mean=74 mph). What is interesting is the lack of effect of D₃F_{A}C_{C}P_{G} at the first test interval at the end of the injury period—no significant increase in serve velocity. In contrast, there was a large increase in velocity in the second test period at the end of the D₃F_{A}C_{C}P_{G} treatment (mean=95.5 mph).

### Leg Press Strength in Four Temporarily Injured Subjects

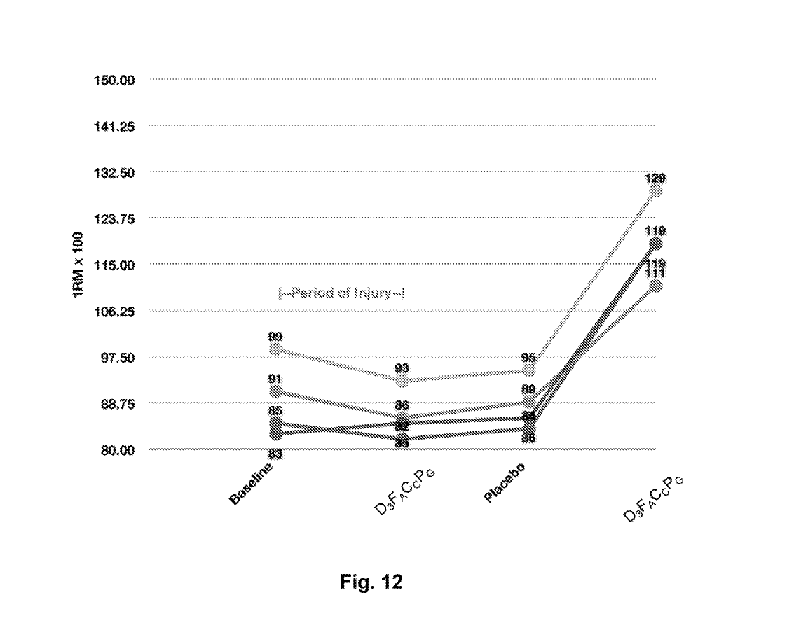

Leg Strength Results: FIG. 12 depicts leg strength measurements from each of the injured subjects. Baseline leg strength was similar to the rest of the group (mean=89.5). Again, what is interesting is the lack of effect of D₃F_{A}C_{C}P_{G} at the first test interval at the end of the injury period—no increase in leg strength. In contrast, there was a large increase in measured leg strength at the second test (mean=119.5).

### Discussion

Prevented from engaging in relevant sport-related activity when taking D₃F_{A}C_{C}P_{G}, which is what occurred with these injured subjects, D₃F_{A}C_{C}P_{G} has no performance effects. When the same subjects recover and can engage in relevant sport-related behavior, D₃F_{A}C_{C}P_{G} leads to measured enhancements in performance. D₃F_{A}C_{C}P_{G} does not affect sport related behavior in a non-specific manner. In contrast, activation of sport-relevant muscle groups leads to a broadly defined process of use-dependent plasticity involving fast-twitch muscle fiber groups. The enhanced fast twitch fibers would be expected to produce more explosive muscle responses that would be precursors to a greater velocity tennis serve and more powerful and explosive leg muscle extensor groups as would be beneficial for leg press strength.

### Example 5

### Additional Data: Leg Press/Standing Vertical Jump

FIG. 13 is a summary figure showing tennis serve velocity, leg press, standing vertical jump and body weight measured at baseline and after 90 days of D₃F_{A}C_{C}P_{G} for the 35-51 yr old male tennis players. FIG. 13 depicts the raw score for each measure (inside the bar) and the percent increase from baseline after the first 90 day treatment with D₃F_{A}C_{C}P_{G} for serve velocity, 1RM leg press and standing vertical jump. The body weight measures were taken at baseline, then approximately 270 days later at the end of the experiment after the subjects had reported weight loss. The correlation between percent change in leg press and standing vertical jump was r(19)=0.68, p<0.001 indicating that there is a relationship between the two measures with greater change in leg strength possibly facilitating vertical jump performance.

The body weight change observed between initial baseline and the conclusion of the study is shown in the last two bars of FIG. 13. The average weight of the subjects was 203 lbs. at the start of the study and over the course of 270 days the average weight decreased approx. 9+ lbs. to 194 lbs. The 4.6% decrease was subjected to a post-hoc test of the difference between the means and the test approached significance but was not so, p=0.08.

### Example 6

### Individual Differences: Tennis Serve Velocity for Each of the Twenty Subjects (Baseline to First Test after 90 Days D₃F_{A}C_{C}P_{G})

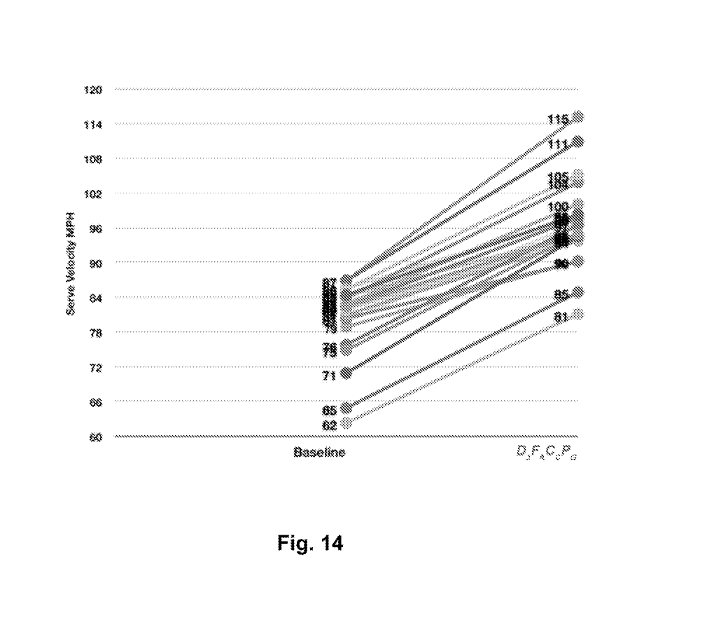

Individual differences with respect to the magnitude of the response to D₃F_{A}C_{C}P_{G} are present in all the studies conducted to date. Because all subjects vary in baseline ability, talent, training, experience and a myriad of other relevant factors, there will be individual differences within just about any measured variable or response. FIG. 14 as an example of baseline individual differences (or differential susceptibility to D₃F_{A}C_{C}P_{G}) among subjects observe the individual variability in tennis serve velocity and the magnitude of the response after D₃F_{A}C_{C}P_{G}. BASELINE: There was variability among the subjects on baseline serve velocity, range 62 to 87 mph. TEST: Each subject showed an increased serve velocity from baseline to the first test period. There were individual differences with respect to the magnitude of the effect of D₃F_{A}C_{C}P_{G}. The percentage increase ranged from a low of +12.1% to a high of +33.2%, with the average increase for the group being +21.4. The correlation between initial serve velocity and D₃F_{A}C_{C}P_{G} enhanced serve velocity was positively related, r(19)=+0.768, p<0.01. Indicating that those subjects that served relatively faster at baseline also served relatively faster after D₃F_{A}C_{C}P_{G}.

While there may be a variety of individual factors that may affect the absolute magnitude of a subject's response to D₃F_{A}C_{C}P_{G}, every subject showed an increase in serve velocity.

### Example 7

### Effect of Varying the Dosage of Vitamin D in D₃F_{A}C_{C}P_{G} on Standing Vertical Jump Performance in Young Adult Males: Inverted U-Shaped Dose Response Function

This experiment examined the effects of a range of doses of vitamin D in D₃F_{A}C_{C}P_{G} on the standing vertical jump. In this study, healthy male subjects were recruited as part of an experiment to examine the effects of nutritional supplements on athletic performance. They were all college-age student participants in a regional summer athletic league. The average age was 20.4 yrs (range 18-22.4 yrs). A total of 66 subjects participated in the study of which data for 60 was recovered. A baseline measure of standing vertical jump was obtained and the subjects were assigned to one of six groups such that the average baseline standing vertical jump of the groups was matched.

There were six groups of 11 subjects each. After matching for standing vertical jump, one group was randomly selected to receive the placebo; the other five groups were randomly selected to receive graded incremental concentrations of vitamin D less than and greater than the dose in D₃F_{A}C_{C}P_{G}. The placebo was a sugar pill. Vitamin D₃ dosage groups were: 400 IU; 2500 IU; D₃F_{A}C_{C}P_{G}=5150 IU; 7500 IU and 10,000 IU. After a 90 day period of treatment a TEST measure of standing vertical jump was obtained. There was a single measure of standing vertical jump recorded after appropriate warm-up.

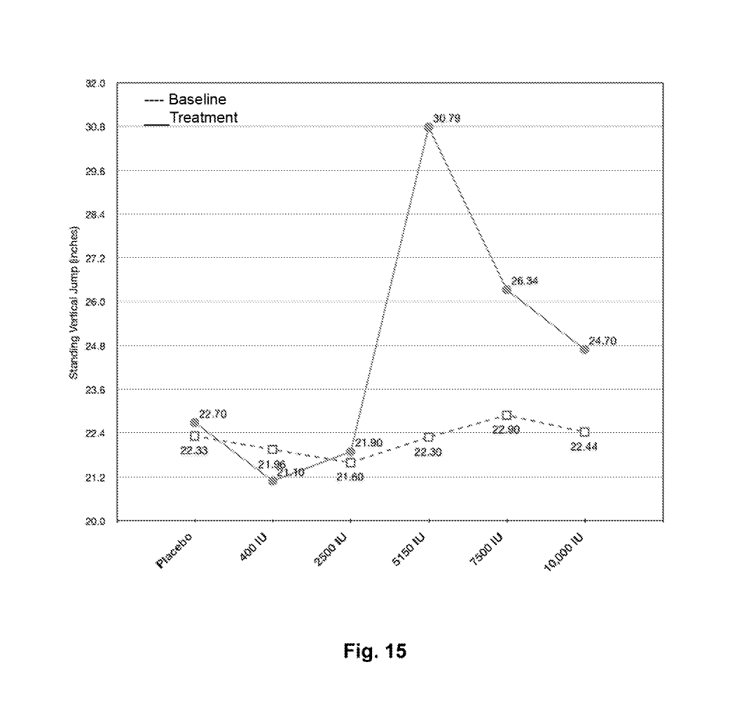

Results. One subject from three separate groups did not complete the test measure. Since the groups were matched on baseline jumping ability, the data from one randomly selected subject from the remaining groups was eliminated leaving an n of 10 per group. The group mean standing vertical jump for BASELINE and TEST is shown in FIG. 15. An analysis of variance showed a significant Group effect, p<0.01, indicating that some groups jumped higher than others as well as a Group x Treatment interaction, p<0.01, reflecting the differential increase in standing vertical jump among the groups. Indicating that not all groups were equally affected. Planned comparisons of the means showed that for the first 3 groups (Placebo, 400 IU, 2500 IU) that the TEST standing vertical jump was not different than the BASELINE standing vertical jump, all p's>0.50. In contrast, the other three remaining groups the TEST standing vertical jump was significantly higher than the BASELINE standing vertical jump and each group was significantly different from the other. Comparison of the TEST means of group D₃F_{A}C_{C}P_{G} with 7500 IU and 10,000 IU showed that D₃F_{A}C_{C}P_{G} standing vertical jump was greater than 7500 IU, p<0.01 and 10,000 IU, p<0.01. 7500 IU also resulted in a greater standing vertical jump than did 10,000 IU, p<0.05.

### Discussion

Inverted U-shaped dose response function. There is an optimal concentration of vitamin D in D₃F_{A}C_{C}P_{G} associated with potentiated standing vertical jump performance in this study. The two lower doses, 400 IU and 2500 IU, did not induce any change in standing vertical jump performance compared to placebo. In contrast, the concentration of vitamin D in D₃F_{A}C_{C}P_{G} resulted in the highest observed standing vertical jump performance and with increasing concentrations of vitamin D (7500 IU and 10,000 IU) recorded standing vertical jump performance decreased. It is interesting to note the almost all-or-none effect on performance between 2500 IU and D₃F_{A}C_{C}P_{G}. This observation suggests that the effect on performance could be dependent on the achievement of a threshold and/or maintenance of tonic optimal levels of activated vitamin D 1,25(OH)₂D₃. Taking more or less than the optimal dose of vitamin D results in less than optimal or no improvement in vertical jump performance. The dose-response function related to the amount of vitamin D in D₃F_{A}C_{C}P_{G} and the effect on standing vertical jump performance is depicted in FIG. 15. At placebo, 400 IU and 2500 IU there is no change in standing vertical jump. At 5150 IU, the maximal response is observed, which drops off with increasing dosage. Between 2500 IU and 5150 IU is the threshold for the potentiated standing vertical jump. Between 2500 IU and 7499 IU is the optimal dose of vitamin D.

The mechanism producing the enhanced performance is of genomic origin (protein synthesis) rather than non-genomic (calcium and phosphate transfer) as would be expected from calcium or potassium deficiencies. That is, because the placebo and two lower doses contained calcium, potassium and folic acid and no performance effects were observed, it is unlikely that the observed performance effects of the 5150 IU and 7500 IU were due to calcium, potassium and/or folic acid. Since vitamin D affects over 1000 human genes, functions as a broadly defined molecular switch and is involved in de novo protein synthesis that leads to an increase of type II muscle fibers, it is reasonable to conclude that the performance effects were due to either a threshold effect of vitamin D on gene transcription or an interaction between attainment of a critical threshold of a vitamin D metabolite and the presence of one or more of the other elements (calcium, potassium and/or folic acid).

### Example 8

### Synergistic Effects of D₃ and Folic Acid on Standing Vertical Jump Performance

This experiment examined pairwise comparisons of the ingredients in D₃F_{A}C_{C}P_{G} on standing vertical jump performance using a within subjects repeated measures design. The subjects were young adult males; players in a competitive basketball league (mean age 20.8 years), recruited through advertisement for a study, the purpose of which was to examine the effects of nutritional supplements on sport performance.

There were five groups, each composed of six subjects. After matching for standing vertical jump, one group was randomly selected to receive the placebo; the other four groups were randomly selected to receive some of possible pairwise combinations of the elements in D₃F_{A}C_{C}P_{G} (Table 2). Subjects ingested the treatment daily for a total of 90 days. At the end of the 90 day period a single measure of standing vertical jump was recorded after an appropriate warm-up period.

TABLE 2
Compositions of the elements comprising
the treatments in Example 8

| GROUP | TREATMENT |
|---|---|
| Placebo | Sugar pill |
| D₃F_{A}C_{C}P_{G} | Vitamin D3 5150 IU, folic acid 400 mcg, calcium |
| | carbonate 600 mg, potassium gluconate 60 mg |
| D₃F_{A} | Vitamin D3 5150 IU, folic acid 400 mcg |
| D₃C_{C} | Vitamin D3 5150 IU, calcium carbonate 600 mg |
| D₃P_{G} | Vitamin D3 5150 IU, potassium gluconate 60 mg |

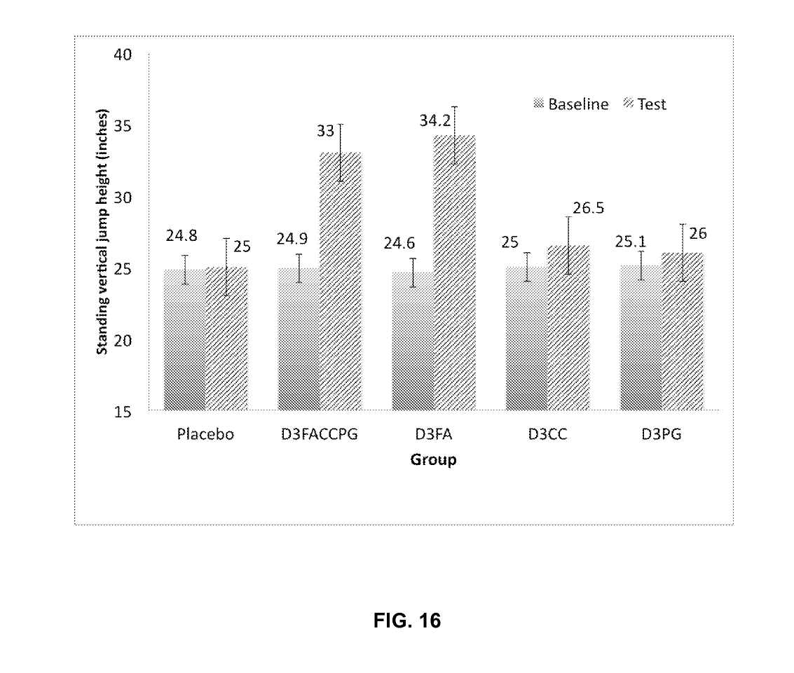

Results. FIG. 16 depicts the baseline and test jump performance for each of the groups. An ANOVA found a significant Group effect, p<0.05 indicating that some groups jumped higher than others as well as a Group x Treatment interaction p<0.05, indicating a differential increase in jump height among the groups. Planned comparisons showed that the means of the placebo, D₃C_{C}, and D₃P_{G} groups were not different between the baseline and test measures. However, the test jump height of the D₃F_{A}C_{C}P_{G} and D₃F_{A} groups were significantly greater than baseline measures, p<0.01. The D₃F_{A}C_{C}P_{G} and D₃F_{A} groups' test jump heights were not different from each other, p>0.30.

### Discussion

The specific combination of vitamin D3 and folic acid is what is responsible for the enhanced jump performance seen with D₃F_{A}C_{C}P_{G}. The other elements, calcium carbonate and potassium gluconate, may serve supportive roles in the overall effect of D₃F_{A}C_{C}P_{G} however, neither is critical to the observed performance effects. In contrast, the specific pairing of folic acid with vitamin D₃ in D₃F_{A}C_{C}P_{G} appears to be the key step in the function of D₃F_{A}C_{C}P_{G}. As such, given the demonstrated genomic actions of both vitamin D₃ and folic acid (folic acid is a methyl donor required for proper DNA transcription), the present data indicate that both substances synergistically contribute to the upregulation of gene transcription necessary for the development of fast-twitch muscle fiber.

### Example 9

### Relative Distribution of Skeletal Muscle Fiber Types Following D₃F_{A}C_{C}P_{G} Treatment

One mechanism to account for the potentiated athletic-related behaviors (throwing, sprint, jump, tennis serve velocity) following D₃F_{A}C_{C}P_{G} treatment would be a structural change in the skeletal muscles that contribute to these behaviors. To determine if muscle fibers change as a result of D₃F_{A}C_{C}P_{G} treatment, muscle fiber type was examined before treatment and following a 90 day D₃F_{A}C_{C}P_{G} treatment in a subject.

Procedure: The subject was a 49 year old healthy male. The subject was instructed to carry out his normal activity routine, which included interval training and jumping sports (basketball). Skeletal muscle fiber distribution was analyzed from percutaneous needle (5 mm) samples obtained under local anesthesia from the vastus lateralis muscle. Before and after treatment samples were required therefore an attempt was made to extract the samples from approximately the same location of the dominant leg ˜15 cm proximal to the superior extent of the patella from the superficial portion of the vastus lateralis muscle (20 mm deep to the fascia lata). The samples were examined under a magnifying glass to determine fiber orientation and then mounted transversely in embedding medium and frozen. Serial 10-μm thick sections were mounted and stained for myofibrillar ATPase based on the procedures of Perrie and Bumford (1986) to differentiate Type I (slow twitch) from Type II (fast twitch) fibers based on staining intensity. Fiber area and relative proportion of the fiber types were computed from the stained sections using a computer-assisted image-analysis system. Relative fiber type distribution was calculated from an average of 527 fibers in each sample. Two representative samples from the baseline and test phase of the experiment were analyzed. Standing vertical jump performance was assessed before and after D₃F_{A}C_{C}P_{G} treatment.

TABLE 3
Cross-sectional area, percentage and total area of muscle
fibers classified according to myofibrillar ATPase staining
and standing vertical jump height in a subject.

| | TYPE I | | TYPE II | | Total | |
|---|---|---|---|---|---|---|
| | Area | | Area | | Area | Jump |
| | μm² | % | μm² | % | μm² | inches |
| Baseline | 4800 | 37.5% | 8000 | 62.5% | 12,800 | 20.5 |
| Test | 4400 | 27.7% | 11,500 | 72.3% | 15,900 | 28 |

Results. The change in the area occupied by type II fibers was dramatically different from baseline to test. Following 90 days of D₃F_{A}C_{C}P_{G} treatment, the total area of section classified as type II fiber increased over 30% from 8000 μm to 11,500 μm. Type I fiber density decreased about 9%. Behaviorally, standing vertical jump performance increased approximately 36%; from a pretest baseline measure of 20.5 inches to 28 inches post-D₃F_{A}C_{C}P_{G} treatment.

### Discussion

This experiment found that D₃F_{A}C_{C}P_{G} treatment modified the relative distribution of type II fibers vs. type I in human thigh muscle. The muscle tissue sections had the appearance of being more densely packed with type II histochemically reactive fibers. In this respect, the muscle specimens of the 49 year old subject's vastus lateralis following D₃F_{A}C_{C}P_{G} treatment resembled that of a much younger subject.

### Other Uses of the Invention

As the invention's mechanism of operation involves the potentiation of the active hormone 1,25(OH)₂D₃ by D₃F_{A}C_{C}P_{G}, it is to be expected by anyone with ordinary skill in the art to infer that the invention will also have other beneficial effects as well. Any biological process involving vitamin D will be affected by D₃F_{A}C_{C}P_{G}; the following serve as examples that have been tested. The following examples are given for the purpose of illustrating various uses of the invention and are not meant to limit the invention in any fashion.

### Example 8

### Moderate to Severe Plaque Psoriasis

Psoriasis is a hyperproliferative skin disorder affected by vitamin D. Powerful topical pharmacological medications containing vitamin D compounds are effective, in some cases, in controlling symptoms. However, these medications are not without side effects. D₃F_{A}C_{C}P_{G} is effective in treating, and in some cases, curing psoriasis in several subjects. Over the course of taking D₃F_{A}C_{C}P_{G} for related athletic behavior studies, subjects that were afflicted with psoriasis reported that their symptoms disappeared and remained dormant throughout the course of treatment. The benefits of D₃F_{A}C_{C}P_{G} over prior art is that D₃F_{A}C_{C}P_{G} functions at the systemic level affecting all de novo skin cell proliferation not just the skin cells at the site of the topical application of current compounds.

### Example 9

### Ulcerative Colitis

Ulcerative colitis is a disease believed caused by a chronic inflammatory process of either autoimmune or idiopathic origin. In a subject taking D₃F_{A}C_{C}P_{G} it was recorded that active ulcerative colitis symptoms abated within 48 hours of the initial treatment and remained absent as long as the subject took daily administrations of D₃F_{A}C_{C}P_{G}. The benefits of D₃F_{A}C_{C}P_{G} over prior art is that D₃F_{A}C_{C}P_{G} functions at the systemic level affecting all de novo inflammation not just the inflammation at the site of the topical application of current compounds.

### Example 10

### Prostate Cancer

Prostate cancer is a malignant hyperproliferative neoplastic disorder. A subject taking D₃F_{A}C_{C}P_{G}, who also was previously diagnosed with malignant prostate cancer (Gleason score 4), reported the complete abatement of neoplastic cells after administration of D₃F_{A}C_{C}P_{G}. The benefits of D₃F_{A}C_{C}P_{G} over prior art is that D₃F_{A}C_{C}P_{G} functions at the systemic level affecting all de novo inflammation not just the inflammation at the site of the application of current compounds.

### Example 11

### Tanning and Skin Pigmentation

When human skin is exposed to sunlight, melanin production increases and skin darkens. This process is commonly known as tanning. It has been discovered that administration of D₃F_{A}C_{C}P_{G} increases the rate at which skin tans and the duration of said tan is greatly increased without the addition of maintenance doses of sunlight.

### Example 12

### Concussion

The edema and inflammation that follows closed head trauma of the type commonly encountered in contact sports is generally referred to a concussion. It has been discovered that when taking D₃F_{A}C_{C}P_{G} concussive symptoms dissipate at a faster rate.

### REFERENCES

Binkley N. (2007) J. Clinical End Met, 92, 3152-3157.
Birge, S. J. & Haddad, J. G. (1975). J. of Clinical Inv., 56, 1100-1107.
Bischoff, H. A., et al., (2001). Histochem. J., 33, 19-24.
Cannell J, et. al., (2009). Med Sci Sports Exerc, 41, 1102-1110.
Chapuy, M. C. et al., (1997). Osteoporos Int., 7, 439-443.
Cross, H. S. et al., (2006). J. of Nutrition, 136, 561-564.
Freedman, L. P. (1999). J. of Nutrition, 129, 581-586.
Garcia, R. B. & Rodriguez Guisado, F. A. (2011). Nutr. Hosp., 26, 945-951.
Gordon C. et al., (2004). Arch. of Ped. & Adole. Med., 158, 531-537.
Gorkin Z., et al., (1938) Fiziol. Zh USSR, 25, 695-701.
Gollnick P. D. et al., (1972). J. of Appl. Phys., 33, 312-319.
Gollnick P. D. (1973). J. of Appl. Phys., 34, 107-111.
Heaney, R. P. et al., (2003). Amer. J of Clin. Nutr., 77, 204-210.
Holick, M. F. (2003). In Primer on metabolic bone diseases and disorders of mineral metabolism, 5^{th} ed. Washington D.C.: Amer. Soc. Bone & Mineral Res., 129-137.
Holick, M. F. (2007). New. Eng. J. Med., 357, 266-281.
Hollis, B. W., et al., (2007). J. of Ster. Biochem & Mol. Bio., 103, 631-634.
Levis, S., et al., (2005). J. of Clin. Endo. Metab., 90, 1557-1562.
Lichtmann, A. R. (2009). Compound D₃F_{A}C_{C}P_{G} greatly prolongs the half-life of 1,25(OH)₂D₃ in vivo. Personal communication.
Lovell G. (2008). Clin. J. of Sports Med., 18, 159-161.
Perrie, W. T. and Bumford, S. J. (1986). J. Neurol. Sci, 73, 89-96.
Vieth, R., et al., (2001). Amer. J. of Clin. Nutr., 73, 288-294.
Ward K. (2009). J. of Clin. Endo. Metab., 94, 559-563.
Yetley, E. A. (2008). Amer. J. of Clin Nutr., 88, 558-564.

Any patents or publications mentioned in this specification are indicative of the level of those skilled in the art to which the invention pertains. Further, these patents and publications are incorporated by reference herein to the same extent as if each individual publication was specifically and individually incorporated by reference.

One skilled in the art would appreciate readily that the present invention is well adapted to carry out the objects and obtain the ends and advantages mentioned, as well as those objects, ends and advantages inherent therein. Changes therein and other uses which are encompassed within the spirit of the invention as defined by the scope of the claims will occur to those skilled in the art.

What is claimed:

1. A composition comprising a first amount of a biologically active vitamin D₃ compound cholecalciferol, a second amount of folic acid, a third amount of calcium carbonate (36% calcium), and a fourth amount of potassium gluconate (16% potassium) or pharmaceutical compositions thereof.

2. The composition of claim 1, wherein said first amount is between 2500 IU-7500 IU.

3. The composition of claim 1, wherein said second amount is between 100 mcg-1000 mcg.

4. The composition of claim 1, wherein said third amount is between 200 mg-1200 mg.

5. The composition of claim 1, wherein said fourth amount is between 20 mg-120 mg.

6. The method of claim 1, wherein fast twitch (type II) skeletal muscle fiber in a subject is increased, comprising administering a therapeutically effective amount of the composition of claim 1.

7. The method of claim 1 wherein the preferred embodiment is administered to a subject in the form of a powdered tablet.

8. The method of claim 1 wherein the preferred embodiment is administered to a subject in the form of a water-based solution.

9. The method of claim 1 wherein the preferred embodiment is administered to a subject in the form of a solid food bar.

10. The method of claim 1 wherein the preferred embodiment is administered to a subject in the form of a transdermal patch.

11. The method of claim 1 wherein the preferred embodiment is administered to a subject in the form of a oil-based gel capsule.

12. The method of claim 1 wherein the preferred embodiment is part of a kit comprising instructions for use.

13. The method of claim 1, wherein the preferred embodiment is administered once per day for 90 consecutive days, and daily henceforth.

14. The method of claim 1, wherein the subject, in conjunction with administration of the preferred embodiment of the composition, performs, practices and/or trains the target motor tasks or skills to be potentiated, enhanced and/or strengthened.

15. The method of claim 1, wherein skeletal muscular strength as measured by, but not limited to, pitched baseball velocity, running speed, standing vertical jump height, tennis serve velocity, and leg extension strength is increased in a subject, comprising administering a therapeutically effective amount of the composition of claim 1.

16. The method of claim 1, wherein the skeletal muscle strength gains return to pre-administration levels about 90 days after cessation of administration of the composition in claim 1.

17. The method of claim 1, wherein body fat and/or body mass index is decreased and/or reduced in a subject, comprising administering a therapeutically effective amount of the composition of claim 1.

18. The method of claim 1, wherein melanin content is maintained or increased in the skin of a subject, comprising administering a therapeutically effective amount of the composition of claim 1.

19. A method for inhibiting proliferation of a cell, comprising contacting a cell with one or more compounds identified in claim 1.

20. The method of claim 19, wherein the cell is in vivo and is associated with a pathophysiological condition in a subject.

21. The method of claim 19, wherein the condition is a skin or mucosal disorder or a defect in cell differentiation.

22. The method of claim 21, wherein the skin disorder is a hyperproliferative skin disorder, a pigmentary skin disorder, an inflammatory skin disorder, or other skin disorder characterized by hair growth on legs, arms, torso, or face, or alopecia, or skin aging, skin damage or a pre-carcinogenic state.

23. The method of claim 21, wherein the hyperproliferative skin disorder is psoriasis or a keloid or fibromatosis, the pigmentary skin disorder is vitiligo, the inflammatory or autoimmune skin disorder is pemphigus, bullous pemphigiod, allergic contact dermatitis, atopic dermatitis, acne vulgarus, or lupus erythematosus.

24. The method of claim 20, wherein the condition is associated with undifferentiated cells or defectively differentiated cells, said contact further inducing differentiation thereof.

25. The method of claim 19, wherein the autoimmune disease or inflammatory process is scleroderma or morphea, keloid or fibromatosis, rheumatoid arthritis, multiple sclerosis, inflammatory bowel disease, concussion, Crohn's disease, interstitial cystitis, or diabetes.

26. The method of claim 19, wherein a cell is a normally proliferating or abnormally proliferating adrenal cell, gonadal cell, keratinocyte or melanocyte, pancreatic cell, cell from the gastrointestinal tract, prostate cell, breast cell, lung cell, immune cell, hematologic cell, kidney cell, brain cell, cell of neural crest origin, skin cell, mesenchymal cell, leukemia cell, melanoma cell, or osteosarcoma cells.

27. The method of claim 1, wherein the loss of skeletal muscle fibers as is associated with sarcopenia in a subject is decreased or halted, comprising administering a therapeutically effective amount of the composition of claim 1
